SEQUENCE LISTING (1 sequence)

SEQ ID NO: 1 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

(19) United States
(12) Patent Application Publication
(10) Pub. No.: US 2011/0229970 A1
(43) Pub. Date: Sep. 22, 2011

(54) DUAL-CHAMBER PERFUSION BIOREACTOR FOR ORTHOPEDIC TISSUE INTERFACES AND METHODS OF USE

(75) Inventor: TENG MA, Tallahassee, FL (US)

(73) Assignee: Florida State University Research Foundation, Tallahassee, FL (US)

(21) Appl. No.: 13/041,688

(22) Filed: Mar. 7, 2011

Related U.S. Application Data
(60) Provisional application No. 61/311,007, filed on Mar. 5, 2010.

(51) Int. Cl. C12N 15/85; C12N 5/071; C12N 5/0775; C12M 3/00; B82Y 5/00

(52) U.S. Cl. 435/455; 435/397; 435/395; 435/303.1; 977/773; 977/795; 977/902

(57) ABSTRACT

The subject invention concerns a perfusion bioreactor device and methods of using the same. A bioreactor device of the invention can be used to grow cells and tissue in a controlled in vitro environment. A perfusion bioreactor device of the invention can have multiple perfusion chambers that can be controlled individually. Transverse or parallel flow of a fluid can be provided to each chamber. Cells can be seeded on a hydrogel and/or 3D scaffold to provide a 3D environment in the bioreactor device where the cells can adhere, proliferate, migrate, secrete growth and/or differentiation factors, and/or undergo differentiation, etc. The subject invention also concerns hydrogels and 3D scaffolds that can be used to grow and/or differentiate cells thereon.

### CROSS-REFERENCE TO RELATED APPLICATION

The present application claims the benefit of U.S. Application Ser. No. 61/311,007, filed Mar. 5, 2010, which is hereby incorporated by reference herein in its entirety, including any figures, tables, nucleic acid sequences, amino acid sequences, and drawings.

### GOVERNMENT SUPPORT

This invention was made with government support under grant number W81XWH-07-1-0363 awarded by the Department of Defense Peer Reviewed Medical Research Program. The government has certain rights in the invention.

### BACKGROUND OF THE INVENTION

Osteoporosis and bone-related diseases and injuries are major public health threats for over 44 million Americans. Osteoporosis is characterized by excessive loss of bone and micro-architectural deterioration of bone tissue leading to bone fragility and increase susceptibility to fractures of hip, spine, and wrist. An overall imbalance between osteoblast-mediated bone formation and osteoclast-mediated bone resorption is believed to the primary cause. One out of two women and one in four men over 50 suffer from the osteoporosis-related fracture and many more are at risk. It is estimated that the annual direct expenditures for osteoporosis and bone related diseases total over $14 billion in 1995 and are increasing as the population ages. Physiotherapy, hormonal replacement therapy, rehabilitation, and orthopedic surgery are the treatment for various forms of osteoporosis at different stages. For site-specific bone defect or fracture repair, autologous bone grafts are the “gold standard” of treatment that provides both structural and functional replacement of host tissue. However, autologous bone grafting is associated with the limitation of harvestable bone, significant morbidity, malformation, and subsequent loss of graft functions. Allograft bone from cadaveric donors is also used but has less osteoinductivity than autogenous bone and immune response and viral infection remain a concern. Artificial bone grafts regenerated by combining patient's own cells and biomaterials represent a novel approach that overcomes the donor limitation, reduce immune response, and increase the efficacy for defect repair and healing.

Human mesenchymal stem cells (hMSCs) have gained increasing interest in bone regeneration and repair, owing to their availability, ease of expansion and proven capability of osteogenesis (Salgado et al., 2006; Bernardo 2007). Originally isolated from bone marrow but now identified in multiple tissue sources, MSCs are multipotent progenitor cells responsible for the repair and regeneration of mesenchymal tissue such as bone, cartilage, fat and muscle (Caplan, 2007; Kern et al., 2006). Along with considerable in vitro studies, autologous bone marrow-derived MSCs have also been used in treatments of various bone diseases and demonstrated their therapeutic potential in patients (Horowitz et al., 1999, 2002; Whyte et al., 2003; Tseng et al., 2008). As hMSCs become the cell of choice in bone tissue repair and regeneration, combining hMSCs with scaffolds to augment their bone regeneration potential is an important approach to enhancing the therapeutic outcome. The success of this approach increasingly relies in understanding and controlling the multiple molecular and physical regulatory signals mediated by the 3D constructs.

Biomimetic scaffolds not only serve as three-dimensional (3D) templates for tissue growth, they guide, through chemical and physical characteristics, the molecular milieu to direct stem cell fate (Seib et al., 2009; Zhao et al., 2006; Grayson et al., 2004). It is therefore advantageous in this approach to select a scaffolding material to mimic natural tissue composition in addition to promoting hMSC proliferation and differentiation. Chitosan, gelatin and hydroxyapatite in various combinations are among frequently studied biomimetic composite scaffolds for bone regeneration because of their chemical similarity to natural bone extracellular matrix (ECM) (Zhao et al., 2006; Peter et al., 2010; Kim et al., 2006; O'Brien et al., 2005). Chitosan, a linear polysaccharide, is composed of glucosamine and N-acetyl glucosamine units linked by β(1-4)-glycosidic bonds. Structural similarity of chitosan with various glycosaminoglycans (GAGs) found in the extracellular matrix of bone and cartilage has made chitosan an attractive material in bone and cartilage tissue regeneration. The cationic nature of chitosan allows for mimicking the ECM-rich environment of hone tissue through the formation of insoluble ionic complexes with anionic molecules, such as growth factors, glycosaminoglycans and proteoglycans, which benefit cell growth and tissue formation (Amaral et al., 2005). Gelatin is a partially denaturalized collagen and retains moieties that facilitate cell adhesion and influence cell behaviors (Sionkowka et al., 2004; Taravel and Domard, 1996). Interactions with growth factors are mediated by the abundance of functional groups in gelatin, forming a favorable microenvironment for tissue regeneration. Hydroxyapatite (HA) is the main mineral component of natural bone ECM and has been used to improve biocompatibility and hard tissue integration by sequestering serum proteins (Kilpadi et al., 2001). The presence of HA improves protein adsorption in porous HCG scaffolds and enhances both hMSC long-term growth and osteogenic differentiation upon induction (Zhao et al., 2006).

Apart from a scaffold's biochemical properties, construct fabrication processes such as dynamic cell seeding and cultivation in a bioreactor system play important roles in directing cell-material interactions and influencing construct properties (Zhao et al., 2009; Ding et al., 2008; Li et al., 2001; Sikavitsas et al., 2003). Understanding the interplay between the molecular milieu and physical regulatory factors, such as convective flow and mechanical forces, has the potential to guide stem cell function through interactive regulation of the stem cell microenvironment. Pore size and pore structure of bone scaffolds are known to significantly influence cellular behaviors and bone construct properties, both in vitro and in vivo (Roy et al., 2003). Understanding and modification of the pore structure of scaffolds has been a focus of bone tissue engineering for a wide range of scaffolds (Jones et al., 2009). To improve cell-seeding efficiency and distribution, perfusion cell seeding has been widely used in porous scaffolds, including chitosan-based sponges (Li et al., 2001; Ding et al., 2008; Alvarez-Barreto et al., 2007). Ding et al. (2008) reported perfusion seeding of dermal fibroblasts in collagen-chitosan sponges prolonged cell proliferation, increased cell density and promoted the formation of a more homogeneous construct with less contraction. Compared to cell seeding in 3D poly(ethylene terephthalate) (PET) fibrous scaffolds, chitosan-gelatin constructs are able to capture more cells at higher superficial velocities, owing to the increased tortuosity and cell interception. In contrast to the effort in perfusion cell seeding and cultivation, much less understood is the role of perfusion in modulating the cellular microenvironment of 3D scaffolds. As the adhesion and spatial distribution of the regulatory macromolecules in the 3D scaffolds are dictated by both scaffold structure and perfusion flow, understanding their interplay will provide important insight in engineering a stem cell microenvironment for functional construct development. Examples of 3D culture systems have been described in U.S. Pat. Nos. 6,875,605; 6,943,008; and 7,122,371.

### BRIEF SUMMARY OF THE INVENTION

The subject invention concerns a porous hydrogel or 3D scaffold and a perfusion bioreactor device and methods of using the same. A bioreactor device of the invention can be used to grow cells and tissue in a controlled in vitro environment. A perfusion bioreactor device of the invention can have multiple perfusion chambers that are connected by a porous scaffold and that can be controlled individually. Transverse or parallel flow of a fluid can be provided to each chamber. In one embodiment, cells can be seeded on a porous hydrogel and/or 3D scaffold to provide a 3D environment in the bioreactor device where the cells can adhere, proliferate, migrate, secrete growth and/or differentiation factors, and/or undergo differentiation, etc. In one embodiment, a hydrogel and/or 3D scaffold comprises hydroxyapatite-chitosan-gelatin (HCG) compound.

In a specific embodiment, the hydrogel or scaffold is impregnated or coated with a nucleic acid encoding one or more desired proteins. Cells in the hydrogel or scaffold are transfected with the nucleic acid and express the protein encoded by the nucleic acid. In one embodiment, the scaffold is impregnated or coated with naked nucleic acid. In another embodiment, the scaffold is impregnated or coated with a chitosan-based nanoparticle that encapsulates the protein-encoding nucleic acid. In one embodiment, the protein is a protein involved in growth and/or differentiation of a cell. In an exemplified embodiment, the protein is a bone morphogenic protein (BMP), such as BMP-2.

A major advantage of the present invention is the ability to control the biomechanical and physiochemical conditions in the bioreactor growth chambers individually and the ability to modulate the interactions and communication between two compartments by directing flow. Large tissue constructs require a controlled heterogeneous environment to grow. The current bioreactor technology typically creates a homogenous growth environment by introducing media flow in one direction and is not able to control the communication between different regions of the construct. Recent studies have shown the interactions and communications between the cells in different regions of the construct is a critical factor that determines the eventual properties of the engineered bone. A device of the present invention has the ability to control the flow conditions inside the construct, and thereby influence cell behavior throughout the entire construct. In addition, devices of the invention also enable one to differentiate human stem cells into different cell types within the same construct, e.g., chondrocytes and osteoblasts, and thereby create osteochondral constructs that are composed of both cartilage and bone.

### BRIEF DESCRIPTION OF THE DRAWINGS

FIGS. 1A-1D. From left: Surface of HCG hydrogel before preincubation (FIG. 1A), and after preincubation for 7 days in αMEM+10% FBS (FIG. 1B), showing protein deposits and nanopores, surface of 3D scaffold (FIG. 1C), cross section of 3D scaffold (FIG. 1D), indicating a well defined interconnected pore structure.

FIGS. 2A-2C show HCG hydrogel cell growth kinetics and surface morphology on HCG hydrogel without preincubation (FIG. 2A), after 7 days preincubation in a MEM+10% FBS (FIG. 2B), and cell penetration depth by confocal microscopy on preincubated hydrogels (FIG. 2C).

FIGS. 3A and 3B. Osteoinduction of hMSCs cultured for 28 days on HCG hydrogel is confirmed by the characteristic alkaline phosphatase (ALP) peak (FIG. 3A), and q-PCR data (FIG. 3B), demonstrating the upregulation of osteo-specific markers from TCP cultured hMSC control.

FIGS. 4A-4D. From left, chitosan-DNA nanoparticles are fabricated using GFP-BMP2 plasmid DNA (FIG. 4A). SEM nanoparticle morphology (FIG. 4B). Plasmid protection confirmed by digestion with DNase followed by NP dissociation and electrophoresis (FIG. 4B). GFP expression at 24 hours post nanoparticle administration to TCP cultured hMSCs (FIG. 4C). GFP expression at 72 hours post nanoparticle administration to TCP cultured hMSCs (FIG. 4D).

FIGS. 5A-5C. The effects of substrate mediated transfection, by naked or chitosan nanoparticle encapsulated GFP-BMP2 plasmid DNA on the cell growth kinetics (FIG. 5A), alkaline phosphatase activity (FIG. 5B) and q-PCR mRNA expression of stem cell markers, bone markers, and transfected GFP expression (FIG. 5C).

FIG. 6 shows flow of media within a chamber of the in house perfusion bioreactor system. The system features modular perfusion chambers with multiple sampling ports and has the capability for each chamber to be controlled individually and set for transverse (top) or parallel (bottom) flow, however, all chambers share the same media source and inoculum, facilitating comparison of various operating conditions (Zhao (2005); Zhao (2007)).

FIGS. 7A and 7B. After 21 days culture in the perfusion bioreactor system at 0.1 mL/min under transverse flow conditions SEM images show hMSCs in intimate contact with the HCG scaffold (FIG. 7A) and even surface distribution (FIG. 7B).

FIGS. 8A-8C. Osteogenic differentiation of hMSCs, cultured for 21 days in the perfusion bioreactor at 0.1 mL/min under transverse flow, then removed and placed in osteoinductive media for 35 days is confirmed by Von Kossa staining of 8 μm thick sections from a top scaffold (FIG. 8A), bottom scaffold (FIG. 8B) and control (FIG. 8C). Control was cultured in α-MEM for 35 days after being removed from the bioreactor.

FIGS. 9A and 9B. Schematic of modified depth filtration system used for preconditioning. Three porous HCG scaffolds were held in place by a stainless steel mesh ring and the medium was either held statically (FIG. 9A) or perfused through (FIG. 9B) and recycled at a rate of 0.01 ml/min for 7 days.

FIGS. 10A-10C. SEM images from cross sections of HCG80 (FIG. 10A), HCG50 (FIG. 10B) and HCG20 (FIG. 10C), all at ×400 magnification.

FIGS. 11A-11E. Effect of flow rate (FIG. 11A), pore size (FIG. 11B) and preconditioning (FIG. 11C) on cell-seeding efficiency. Preconditioned HCG80 constructs were used in the flow rate test (FIG. 11A) and the preconditioning test (FIG. 11C). DAPI images show cellular distribution in HCG80 scaffold 6 h after seeding at 1 ml/min (FIGS. 11D and 11E) at ×10 magnification. Constructs were seeded at 2×10⁵ cells (FIGS. 11A-11C) or 2×10⁶ cells/cm³ (FIGS. 11D and 11E). *p<0.05; **p<0.001.

FIGS. 12A-12F. SEM images of preconditioned (FIGS. 12A-12C) and non-conditioned HCG80 scaffolds (FIGS. 12D and 12F). Preconditioning did not change the overall pore structure (FIG. 12A) but created a more fibrous surface texture (FIG. 12B) with the appearance of nanopores (FIG. 12C). Magnifications: (FIGS. 12A and 12D) ×400; (FIGS. 12B, 12C, 12E, 12F) ×30,000.

FIGS. 13A-13D. Protein adsorption characteristics of top, middle and bottom HCG80 scaffolds for VN (FIGS. 13A and 13B) and FN (FIGS. 13C and 13D) after static (FIGS. 13A and 13C) and perfusion (FIGS. 13B and 13D) preconditioning. †The difference is significant (p<0.05) at each time point between all scaffold positions (FIGS. 13B and 13D), except between the middle and bottom VN samples of days 5 and 7. *The differences between top and middle and bottom and middle for all data points under static conditions are also statistically significant. (p<0.05).

FIGS. 14A-14C. Effects of seeding density, pore size and preconditioning on hMSC growth. Cell growth is significantly higher at higher seeding density (500,000 cells/cm³) compared to low (50,000 cells/cm³) initial seeding density in preconditioned HCG80 constructs at all time points (p<0.01) (FIG. 14A). Pore size of preconditioned HCG scaffolds has an insignificant influence on cell growth (FIG. 14B). Preconditioning greatly enhances hMSC growth in HCG80 (FIG. 14C). **p<0.01. FIGS. 15A-15C. Effect of pore size on hMSC osteogenic differentiation on preconditioned samples. Alkaline phosphatase expression is comparable for HCG scaffolds with three different pore sizes (FIG. 15A). Both total and cell based (FIGS. 15B and 15C) OP expression increased from day 14 to 21, whereas OC expression remains comparable for both cell based and total in HCG80. *p<0.05; **p<0.01.

FIG. 16 shows an embodiment of a perfusion bioreactor device of the invention.

### BRIEF DESCRIPTION OF THE SEQUENCES

SEQ ID NO:1 is an amino acid sequence of a human BMP-2 protein.

### DETAILED DESCRIPTION OF THE INVENTION

The subject invention concerns a perfusion bioreactor device and methods of using the same. A bioreactor device of the invention can be used to grow cells and tissue in a controlled in vitro environment. A perfusion bioreactor device 10 of the invention can have multiple perfusion chambers 20 that can be controlled individually. Transverse or parallel flow of a fluid media can be provided to each chamber. Inlet valves 30 and outlet valves 32 can control the flow of fluid media into and out of each chamber. In addition, the parallel flow rate and composition of the fluid media is one that is capable of providing appropriate conditions for cell life and/or supporting and directing growth and/or differentiation of cells within the device. For example, the fluid media can be a fluid containing nutrients and other chemicals or factors, such as serum, glucose, cell growth factors and cytokines, O₂, etc. to support the growth and/or differentiation of cells. In one embodiment, the perfusion bioreactor chamber has two or more compartments 40 connected by one or more porous scaffolds 50 (onto which cells can be seeded and fluid media can flow through), and conditions such as substance concentration, pressure, and fluid flow rate can be individually controlled in each compartment of each chamber. Inlet valves 60 and outlet valves 62 can control the flow of fluid media into and out of each individual compartment. The pressure in each chamber compartment can be regulated so that the fluid can penetrate the scaffold transversely or horizontally on demand. The porous scaffold in the chamber supports cell growth and fluid penetration thereby providing space for the cells to form a functional tissue such as bone, cartilage, or tendon. In one embodiment, the porous scaffold is a porous hydrogel and/or 3D scaffold to provide a 3D environment in the bioreactor device where the cells can adhere, proliferate, migrate, secrete growth and/or differentiation factors, and/or undergo differentiation, etc.

In one embodiment, a hydrogel and/or 3D scaffold comprises or is composed of a hydroxyapatite-chitosan-gelatin (HCG) compound. The hydrogel or scaffold can optionally be provided in a shape that mimics or is similar to the shape of the tissue that is to be repaired or replaced in a human or animal. In addition, the pore size of the hydrogel or scaffold can be controlled and selected to promote formation of a particular tissue type, e.g., bone-like tissue. In one embodiment, the hydrogel or 3D scaffold is preconditioned with a fluid media that supports cell growth and/or differentiation prior to seeding with cells. In a specific embodiment, the fluid media comprises serum. In one embodiment, the serum is fetal bovine serum. In another embodiment, the serum is a human serum. The preconditioning can be performed in a static mode, or in a perfusion mode wherein the fluid media is continuously circulated through the hydrogel or scaffold at a selected flow rate for a selected amount of time. The pore size of the hydrogel or scaffold can also be selected. In one embodiment, the pore size can be varied from about 75 μm to about 250 μm.

A hydrogel or scaffold of the invention can optionally be impregnated or coated with nucleic acid encoding one or more proteins of interest. In one embodiment, the hydrogel or scaffold is impregnated or coated with naked nucleic acid. In another embodiment, the hydrogel or scaffold is impregnated or coated with a nanoparticle that encapsulates the protein-encoding nucleic acid. In a specific embodiment, the nanoparticle is a chitosan-based nanoparticle (Bowman et al. (2006) describe incorporation of nucleic acid in chitosan-based nanoparticles). Cells provided in the hydrogel or scaffold can then be transfected with the nucleic acid impregnated or coated on the hydrogel or scaffold and subsequently express the protein encoded by the nucleic acid. The nucleic acid can be selected to provide one or more particular protein associated with growth and/or differentiation of the cells to a desired tissue type. In one embodiment, the protein encoded by the nucleic acid is a protein involved in growth and/or differentiation of a cell. In an exemplified embodiment, the protein is a bone morphogenic protein (BMP), such as BMP-2. Other BMPs within the scope of the invention include, but are not limited to, BMP-1, BMP-3, BMP-4, BMP-5, BMP-6, BMP-7, BMP-8a, BMP-8b, BMP-9, BMP-10, and BMP-15. In one embodiment, the BMP is a human BMP. In a specific embodiment, a human BMP-2 protein comprises the amino acid sequence shown in SEQ ID NO:1 (GenBank Accession No. AAF21646.1), or a biologically active fragment or variant thereof. Nucleic acid encoding other proteins that can be utilized within the scope of the invention include, but are not limited to, fibroblast growth factors (FGF), vascular endothelial growth factors (VEGF), transforming growth factor beta (TGF-β), and cell density signal molecule (CDS-1) (U.S. Pat. Nos. 5,741,895 and 6,245,899). Nucleic acid sequences encoding the protein of interest are typically known in the art and can be provided in the hydrogel or scaffold of the device for transfection into the cells. Methods for coating or impregnating the hydrogel or scaffold with naked nucleic acid or a nanoparticle that encapsulates the nucleic acid include: plasmid DNA solution at 1-10 μg DNA in 100 μL 5 mM sodium sulfate is added to the bottom of a tissue culture plasma treated plastic plate. HCG solution is added to the same plastic plate at room temperature for 2 hrs before (1) air dry for films or (2) placement in 4° C. refrigerator for 2 hours before transferring to −80° C. freezer for 24 hours followed by lyophilization. Chitosan-DNA nanoparticles at an average particle size of 180 nm with the same DNA concentration is added to the scaffold following the same procedure.

In one embodiment, the cells provided or utilized in the porous hydrogel or scaffold and bioreactor device comprise stem cells. In a specific embodiment, the cells comprise mesenchymal stem cells (MSC). The cells can be human or other mammalian cells.

The subject invention also concerns methods for growing and/or differentiating cells and tissue using a bioreactor device of the invention. Cells are provided in a perfusion chamber of the device and can be seeded on the porous scaffold. In one embodiment, the porous scaffold is a hydrogel and/or 3D scaffold in the chamber. Nutrient media and/or gases (e.g., O₂, N₂, CO₂) is provided to the cells to provide for cell growth and/or differentation. Cells provided in the hydrogel or scaffold can be transfected with a nucleic acid impregnated or coated on the hydrogel or scaffold and subsequently express the protein encoded by the nucleic acid. In one embodiment, the hydrogel or scaffold is impregnated or coated with naked nucleic acid. In another embodiment, the hydrogel or scaffold is impregnated or coated with a nanoparticle that encapsulates the protein-encoding nucleic acid. In a specific embodiment, the nanoparticle is chitosan-based. The nucleic acid can be selected to provide one or more particular protein associated with growth and/or differentiation of the cells to a desired tissue type. In one embodiment, the nucleic acid encodes a protein involved in growth and/or differentiation of a cell. In an exemplified embodiment, the protein is a bone morphogenic protein (BMP), such as BMP-2. Other BMPs within the scope of the invention include, but are not limited to, BMP-1, BMP-3, BMP-4, BMP-5, BMP-6, BMP-7, BMP-8a, BMP-8b, BMP-9, BMP-10, and BMP-15. In one embodiment, the BMP is a human BMP. In a specific embodiment, a human BMP-2 protein comprises the amino acid sequence shown in SEQ ID NO:1 (GenBank Accession No. AAF21646.1), or a biologically active fragment or variant thereof. Nucleic acid encoding other proteins that can be utilized within the scope of the invention include, but are not limited to, fibroblast growth factors (FGF), vascular endothelial growth factors (VEGF), transforming growth factor beta (TGF-β), and cell density signal molecule (CDS-1) (U.S. Pat. Nos. 5,741,895 and 6,245,899). The nucleic acid can be provided in an expression construct. Following growth and/or differentiation of the cells in the bioreactor device, the cells and/or tissue that has been produced can be harvested for use or further culturing. The cells and tissue are maintained in a sterile environment in a bioreactor of the invention so as to minimize contamination by microorganisms. In one embodiment, the cells and/or tissue are differentiated into chondrocytes and/or osteoblasts. In one embodiment, the tissue comprises bone, cartilage, and/or tendon. Other types of cell and tissue constructs are contemplated within the scope of the invention and include, but are not limited to, muscle, skin, vascular, hepatic, etc.

The subject invention also concerns cell and tissue constructs grown and/or produced using a perfusion bioreactor device of the invention. In one embodiment, the construct is a three-dimensional (3D) construct. Tissue constructs can be prepared in a bioreactor device of the invention that contains a scaffolding material. In a specific embodiment, the scaffolding material is a chitosan-based material. In one embodiment, the scaffolding material comprises a hydroxyapatite-chitosan-gelatin compound. The scaffolding material can also be optionally impregnated or coated with a polynucleotide encoding one or more desired proteins that is to be provided and expressed a cell or cells provided in the bioreactor device of the invention. In one embodiment, the hydrogel or scaffold is impregnated or coated with naked nucleic acid. In another embodiment, the hydrogel or scaffold is impregnated or coated with a nanoparticle that encapsulates the protein-encoding nucleic acid. In a specific embodiment, the nanoparticle is a chitosan-based nanoparticle (Bowman et al. (2006) describe incorporation of nucleic acid in chitosan-based nanoparticles). Cells provided in the hydrogel or scaffold can then be transfected with the nucleic acid impregnated or coated on the hydrogel or scaffold and subsequently express the protein encoded by the nucleic acid. The nucleic acid can be selected to provide one or more particular protein associated with growth and/or differentiation of the cells to a desired tissue type. In one embodiment, the protein encoded by the nucleic acid is a protein involved in growth and/or differentiation of a cell. In a specific embodiment, the polynucleotide encodes a bone morphogenic protein or other cellular growth and/or differentiation factors. In an exemplified embodiment, the protein is a bone morphogenic protein (BMP), such as BMP-2. Other BMPs within the scope of the invention include, but are not limited to, BMP-1, BMP-3, BMP-4, BMP-5, BMP-6, BMP-7, BMP-8a, BMP-8b, BMP-9, BMP-10, and BMP-15. In one embodiment, the BMP is a human BMP. In one embodiment, the cell is transformed with a polynucleotide sequence comprising a sequence encoding the BMP-2 amino acid sequence shown in SEQ ID NO:1, or a biologically-active fragment or variant thereof. Nucleic acid encoding other proteins that can be utilized within the scope of the invention include, but are not limited to, fibroblast growth factors (FGF), vascular endothelial growth factors (VEGF), transforming growth factor beta (TGF-β), and cell density signal molecule (CDS-1) (U.S. Pat. Nos. 5,741,895 and 6,245,899). Preferably, the polynucleotide sequence is provided in an expression construct of the invention. Cells in a construct can be differentiated into one or more desired cell types. In one embodiment, cells have been differentiated into chondrocytes and/or osteoblasts to provide an osteochondral construct. In one embodiment, the tissue comprises bone, cartilage, and/or tendon. Other types of cell and tissue constructs are contemplated within the scope of the invention and include, but are not limited to, muscle, skin, vascular, hepatic, etc. The cell and tissue constructs are typically maintained and provided under sterile conditions.

The subject invention also concerns methods for treating a person or animal in need of replacement or repair of tissue, comprising implanting a cell or tissue construct produced using a device and methods of the present invention into a person or animal in need of such treatment. In one embodiment, the cells used to produce the construct are MSC. In a specific embodiment, the cells are human MSC. Methods of the invention can include the steps of removing damaged or diseased tissue from a person or animal and implanting in the person or animal a cell or tissue construct produced using a bioreactor device of the invention. In one embodiment, the tissue construct comprises bone, cartilage, and/or tendon. In one embodiment, the tissue construct can be used in treating osteoporosis or other bone-related diseases or injuries in a person or animal. The tissue construct can be produced in the type of tissue and shape of the tissue that is to be replaced. For example, if the tissue to be replaced is bone, then the tissue construct to be implanted can be produced as bone tissue and in the general shape and size of bone tissue needed for implantation into the person or animal.

A nucleic acid or polynucleotide encoding one or more desired proteins useful in the present invention, such as a BMP, can be provided in an expression construct. Expression constructs that can be used with the invention generally include regulatory elements that are functional in the intended host cell in which the expression construct is to be expressed in. Thus, a person of ordinary skill in the art can select regulatory elements for use in bacterial host cells, yeast host cells, plant host cells, insect host cells, mammalian host cells, and human host cells. In the present invention, expression constructs will generally be constructs for use in mammalian cells, and in particular, human cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the invention can comprise a promoter sequence operably linked to a polynucleotide sequence encoding a desired protein that is to be provided to a cell or cells provided with the bioreactor device of the invention. In one embodiment, the polynucleotide of the expression construct encodes a BMP. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

For expression in animal cells, an expression construct of the invention can comprise suitable promoters that can drive transcription of the polynucleotide sequence. If the cells are mammalian cells, then promoters such as, for example, actin promoter, metallothionein promoter, NF-kappaB promoter, EGR promoter, SRE promoter, IL-2 promoter, NFAT promoter, osteocalcin promoter, SV40 early promoter and SV40 late promoter, Lck promoter, BMP5 promoter, TRP-1 promoter, murine mammary tumor virus long terminal repeat promoter, STAT promoter, or an immunoglobulin promoter can be used in the expression construct.

Expression constructs of the invention may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides of the invention. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent.

DNA sequences which direct polyadenylation of mRNA transcribed from the expression construct can also be included in the expression construct, and include, but are not limited to, an octopine synthase or nopaline synthase signal. Expression constructs can also include one or more dominant selectable marker genes, including, for example, genes encoding antibiotic resistance for selecting transformed cells. Antibiotic-resistance genes can provide for resistance to one or more of the following antibiotics: hygromycin, kanamycin, bleomycin, G418, streptomycin, paromomycin, neomycin, and spectinomycin. Kanamycin resistance can be provided by neomycin phosphotransferase (NPT II). Other markers used for cell transformation screening include genes encoding β-glucuronidase (GUS), β-galactosidase, luciferase, nopaline synthase, chloramphenicol acetyltransferase (CAT), green fluorescence protein (GFP), or enhanced GFP (Yang et al., 1996).

The subject invention also concerns polynucleotide vectors comprising a polynucleotide sequence of the invention that encodes a desired protein that is to be provided to a cell or cells provided with the bioreactor device of the invention. Unique restriction enzyme sites can be included at the 5′ and 3′ ends of an expression construct or polynucleotide of the invention to allow for insertion into a polynucleotide vector. As used herein, the term “vector” refers to any genetic element, including for example, plasmids, cosmids, chromosomes, phage, virus, and the like, which is capable of replication when associated with proper control elements and which can transfer polynucleotide sequences between cells. Vectors contain a nucleotide sequence that permits the vector to replicate in a selected host cell. A number of vectors are available for expression and/or cloning, and include, but are not limited to, pBR322, pUC series, M13 series, and pBLUESCRIPT vectors (Stratagene, La Jolla, Calif.).

Polynucleotides that can be used with the present invention can be composed of either RNA or DNA. Preferably, the polynucleotides are composed of DNA. The subject invention also encompasses those polynucleotides that are complementary in sequence to the polynucleotides disclosed herein.

Because of the degeneracy of the genetic code, a variety of different polynucleotide sequences can encode a desired protein that is to be provided to a cell or cells provided with the bioreactor device of the present invention. In addition, it is well within the skill of a person trained in the art to create alternative polynucleotide sequences encoding the same, or essentially the same, protein. These variant or alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides of the present invention. Allelic variants of the nucleotide sequences encoding a desired protein that is to be provided to a cell or cells provided with the bioreactor device of the invention are also encompassed within the scope of the invention.

Substitution of amino acids other than those specifically exemplified or naturally present in a desired protein that is to be provided to a cell or cells provided with the bioreactor device of the invention are also contemplated within the scope of the present invention. For example, non-natural amino acids can be substituted for the amino acids of a desired protein, so long as the protein having the substituted amino acids retains substantially the same biological activity as the protein in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form. Allelic variants of a protein sequence are also encompassed within the scope of the invention.

Amino acids can be generally categorized in the following classes: non-polar, uncharged polar, basic, and acidic. Conservative substitutions whereby a polynucleotide encoding a desired protein that is to be provided to a cell or cells provided with the bioreactor device of the present invention having an amino acid of one class is replaced with another amino acid of the same class fall within the scope of the subject invention so long as the protein having the substitution still retains substantially the same biological activity as the protein that does not have the substitution. Polynucleotides encoding a desired protein having one or more amino acid substitutions in the sequence are contemplated within the scope of the present invention. Table 1 below provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The subject invention also concerns variants of the polynucleotides of the present invention that encode a desired protein that is to be provided to a cell or cells provided with the bioreactor device of the invention. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes, but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be prepared and tested using standard techniques known in the art.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. Allelic variations of the exemplified sequences also fall within the scope of the subject invention. The polynucleotide sequence includes both the sense and antisense strands either as individual strands or in the duplex.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Materials and Methods for Example 1

HCG hydrogels and scaffolds were fabricated by respective evaporation or sublimation of HCG aqueous suspension (Zhao (2006)). Low passage hMSCs were seeded on HCG hydrogels or HCG 3D scaffolds and cultured statically or in the perfusion bioreactor for a culture period up to 35 days. Cell growth kinetics were analyzed by metabolic assay. Differentiation was analyzed by alkaline phosphatase kinetics and q-PCR. Cell and material morphology were analyzed by epifluorescence, confocal and electron microscopy. Transfection was carried out by incorporating naked or chitosan nanoparticle encapsulated GFP-BMP-2 plasmid DNA into the HCG aqueous suspension, and evaluated by q-PCR. The substrate mediated transfection experimental conditions are illustrated in the schematic below. The in-house modular perfusion bioreactor generates transverse or parallel flow, facilitating dynamic cell seeding and modulating growth microenvironment.

### Example 1

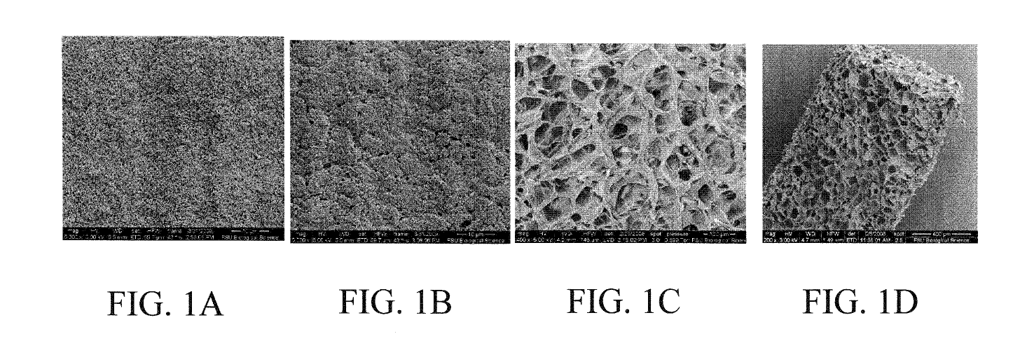

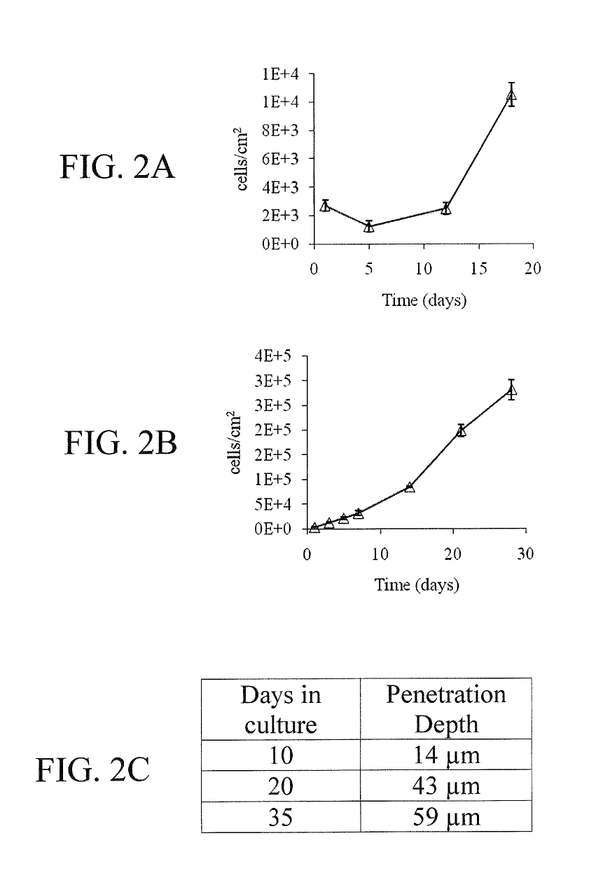

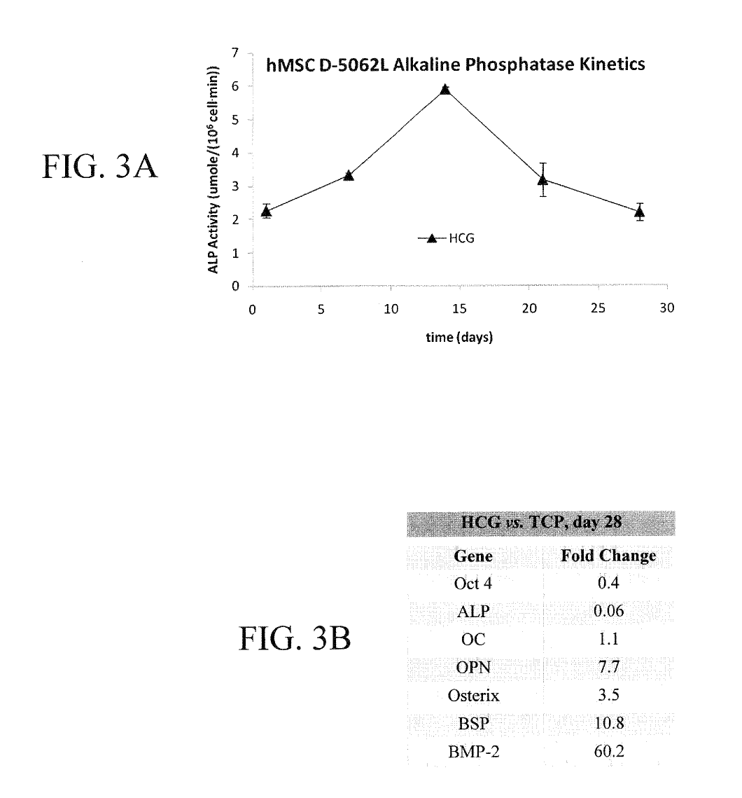

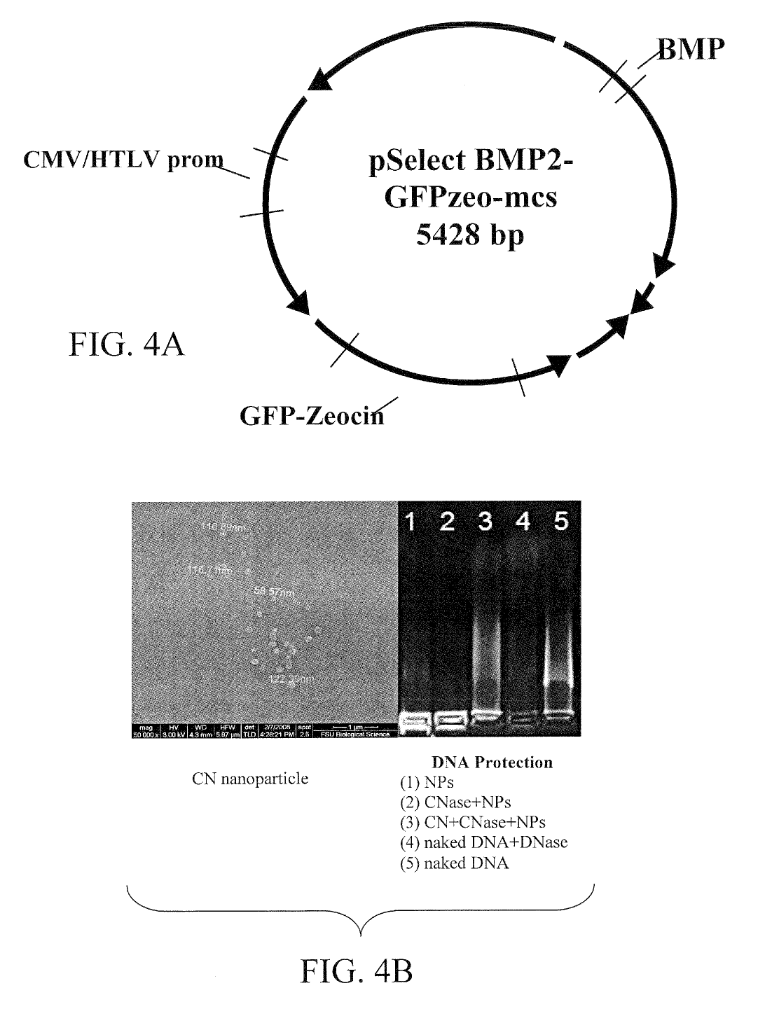

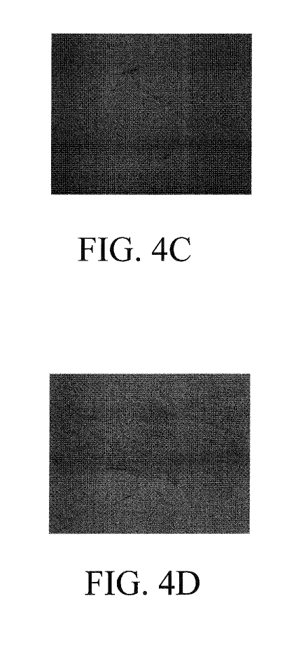

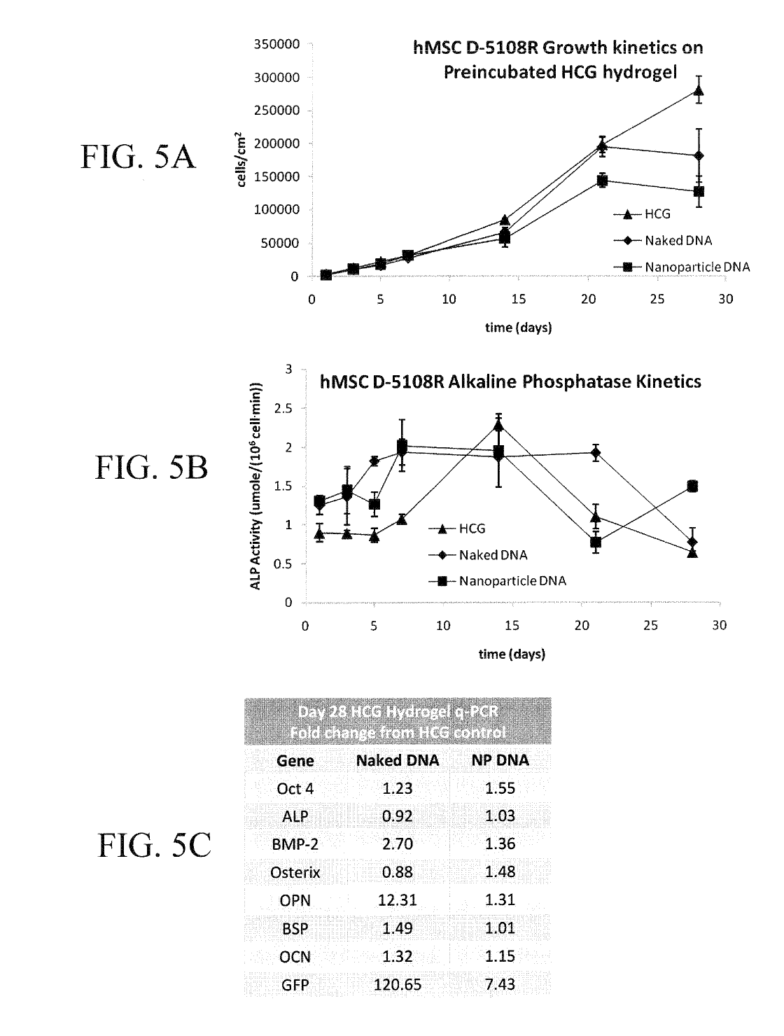

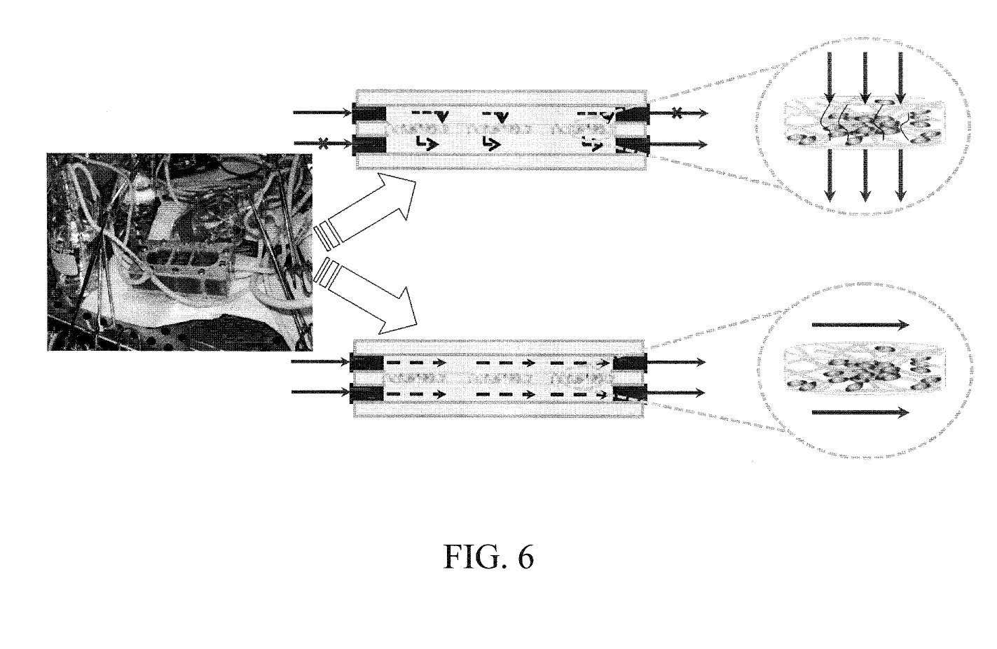

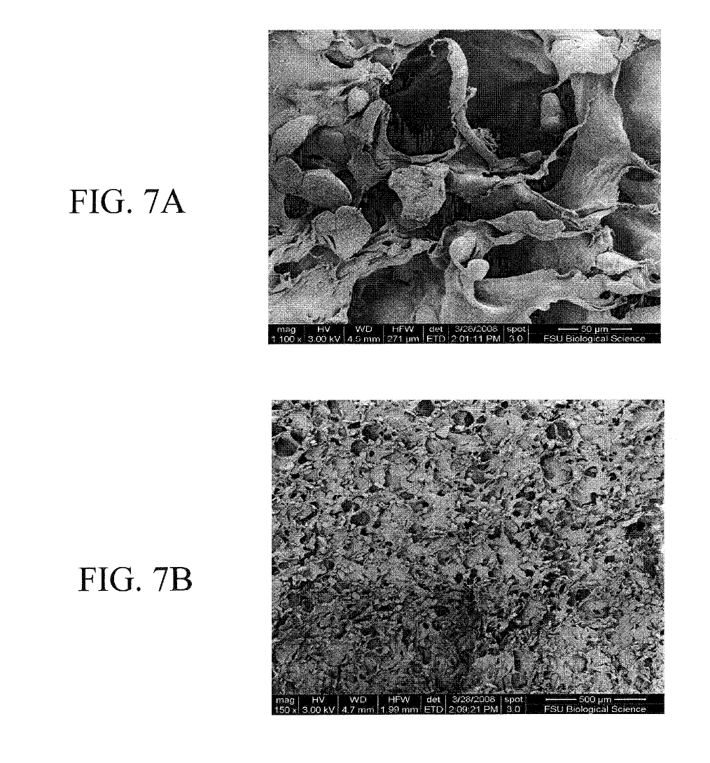

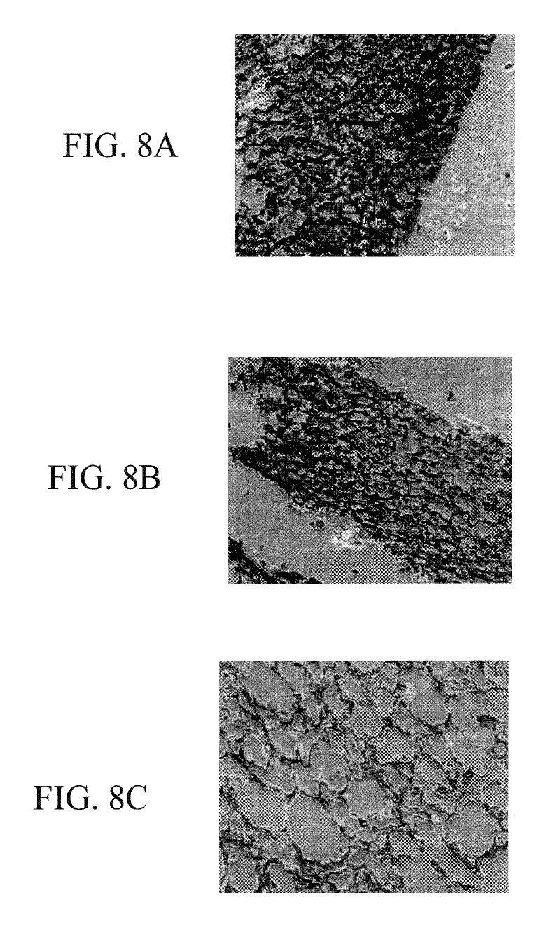

With reference to FIGS. 1-8, hMSC exhibit rapid proliferation in preincubated HCG due to cell migration into the matrices, indicating excellent biomimetic properties HCG's inherent osteoinductive properties without chemical induction is confirmed by hMSC's alkaline phosphatase (ALP) activity and osteogenic gene expression quantified by qPCR. Sustained substrate mediated transfection by incorporating chtiosan-BMP-2 nanoparticles at 28 days of culture is confirmed by q-PCR, demonstrating the potential for achieving sustained gene delivery in HCG matrices. hMSCs maintained their osteogenic differentiation ability after extensive perfusion growth in the HCG scaffolds, indicating the potential of bioreactor for streamlined construct fabrication.

### Materials and Methods for Examples 2-4

Materials. Medium molecular weight chitosan and gelatin from bovine skin were purchased from Sigma Chemical Co. (St. Louis, Mo., USA). Hydroxyapatite (HA) powder with an average particle diameter of 8.99 μm was fabricated and characterized as previously described (Zhao et al., 2002). Chitosan was purified by dissolving in a 2% aqueous acetic acid solution, pH 3. The homogeneous chitosan solution was filtered to remove insolubles and then precipitated by titration with 5 M aqueous ammonium hydroxide, washed repeatedly with deionized (DI) water, frozen and then lyphophilized. All other reagents were obtained from Sigma unless otherwise stated.

Preparation of Porous HCG Scaffolds. Porous HCG Scaffolds were Prepared by solid-liquid phase separation and subsequent sublimation of the solvent, following a method published previously (Zhao et al., 2002, 2006). HA (0.5 g) was weighed into a flask containing 98 ml DI water. The mixture was stirred at room temperature for 0.5 h and treated by ultrasonication until the HA powder was thoroughly dispersed within the DI water. Chitosan (0.625 g) was added into the HA suspension under agitation, followed by the addition of 2 ml acetic acid. The HA-Chitosan solution was left to stir overnight at room temperature to allow the chitosan to fully dissolve. Lastly, 0.625 g gelatin was added to the mixture, then submerged in a 37° C. water bath until fully dissolved. A 1 ml volume of the mixture was then added into each well of a 12-well tissue culture plate. The plate was cooled at 4° C. for 4 h, then transferred to a freezer set to the desired temperature [−80° C. (HCG80); −50° C. (HCG50); or −20° C. (HCG20)] and chilled overnight to induce solid-liquid phase separation. The solidified mixture was transferred to a lyophilizer (Lyo-Centre, VirTis, Warminster, Pa., USA), maintained at the appropriate temperature and freeze-dried for 48 h. Foams were then removed from the lyophilizer and treated with a 10% NaOH solution in aqueous 50% ethanol to neutralize the acetic acid, followed by repeated washings with DI water. The samples were sterilized by immersion in 70% ethanol for 3 h; the ethanol was then removed by soaking for 1 h with three changes of phosphate-buffered saline (PBS).

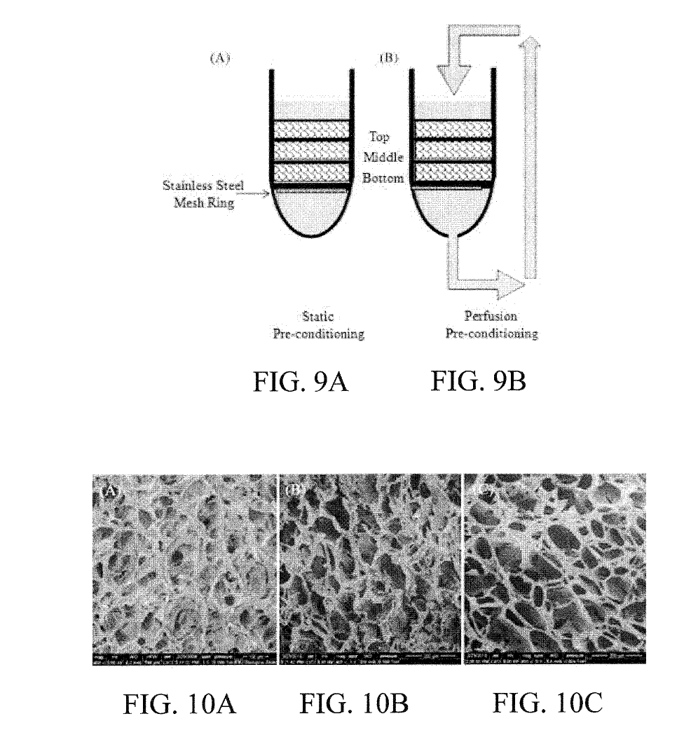

Pre-incubation. Three 1 mm thick sterilized HCG scaffolds were stacked in a custom-made depth filtration device described in our prior publications (Li et al., 2001; Grayson et al., 2004). Complete culture medium, antibiotic-free α-MEM with 10% FBS (Atlanta Biologicals, Lawrenceville, Ga., USA) was added until the 15 ml column was filled and continuously circulated at a flow rate of 0.01 ml/min for 7 days under sterile conditions. For static preconditioning, the scaffolds were placed in the depth filtration device with the same volume of complete culture medium, but no perfusion flow was applied (FIG. 9). The scaffolds were then removed for in situ ELISA or seeded with hMSCs using depth filtration.

Morphology observation and analysis of pore structure. The morphology of the 3D porous scaffolds was examined by scanning electron microscopy (SEM). Scaffolds were either pre-incubated or stored in PBS, fixed in 2.5% glutaraldehyde, frozen in PBS, cross-cut using a scalpel, dehydrated in graded ethanol washings, dried by HMDS evaporation, then mounted and sputter-coated with iridium. Observations were made using a Nova 400 Nano SEM (FEI, Hillsboro, Oreg., USA) under low-vacuum conditions. Three digital images of each longitudinal section and two images of each of the smaller transverse sections were taken and these images were analyzed for each scaffold, using the standard software accompanying the machine. The mean pore diameter of the scaffold at each position within the scaffold was calculated from the average of the mean intercepts in the longitudinal plane and in the adjacent transverse plane.

Cell seeding and culture. Standard frozen human bone MSCs were obtained from the Tulane Center for Gene Therapy and were cultured following a method outlined in our prior publications (Grayson et al., 2004). Briefly, bone marrow aspirates from healthy donors in the age range 19-49 years were collected under an Institutional Review Board-approved protocol. Plastic adherent nucleated cells were separated from the aspirate, expanded on tissue culture grade Petri dishes using complete culture medium, as described above in the Pre-Incubation section, at 37° C. and 5% CO₂, and cultured to passage 5. All cells used in the experiments in this paper were seeded at passage 6.

Cell seeding was performed using the depth filtration method detailed in prior publications (Grayson et al., 2004; Li et al., 2001; Zhao and Ma, 2005). For flow rates of 1.0 ml/min or 30 ml/min, the seeding operation was carried out using the depth filtration device depicted in FIG. 9, while seeding at 0.1 ml/min was carried out in the in-house perfusion bioreactor at the same cell concentration to ensure precise control of flow rate at low range. The detailed description of depth filtration seeding using the bioreactor is described in a prior publication (Zhao and Ma, 2005). Briefly, the perfusion bioreactor system has both seeding and operation loops that are connected to a multichannel precision peristaltic pump (Cole-Palmer, Vernon Hills, Ill., USA). During cell seeding, cell suspension is injected into the bioreactor chamber and perfused through the HCG constructs that are fixed in the centre of the chamber. Three constructs are used in each chamber and four chambers were used in the system. Cell suspension concentration varied by sample and contained between 2×10⁵ and 1×10⁶ cells suspended in 15 ml medium. The suspension was passed through each individual scaffold three times at the respective flow rate to increase the overall seeding efficiency (Li et al., 2001). For flow rates of 1 and 30 ml/min, the seeding operation lasted 5-45 minutes, depending on flow rate, whereas the seeding operation at the perfusion bioreactor lasted 3 h. After seeding, the constructs were transferred to six-well plates, with one construct/well in 3 ml complete culture medium. The medium was changed every 3 days and maintained for up to 21 days at 37° C. and 5% CO₂. To determine seeding efficiency, cells were allowed to adhere for 6 h at 37° C. and 5% CO₂, then the constructs were removed from the well plates, placed in fresh wells and washed three times with sterile PBS, prior to conducting an MTT assay as detailed later in the paper. Seeding efficiency was the ratio of cells present in the initial suspension to the cells present in the construct after 6 h.

Thiazolyl blue tetrazolium bromide (MTT) assay. MTT was obtained from Sigma and dissolved at 5 mg/ml in RPMI Media 1640 (Gibco, Carlsbad, Calif.), 0.8 μm filtered and then stored at −20° C. Culture medium was removed from the samples and cell-seeded constructs were washed with sterile PBS, followed by the addition of antibiotic and phenol-free MEM with 10% FBS at the same volume as the original culture volume. MTT solution, as described above, was added to each well at 10% of the medium volume, and the samples were then incubated at 37° C. and 5% CO₂ for 3 h. After incubation the MTT-containing medium was removed and replaced with an equal volume of 0.1 N HCl in 100% isopropanol under agitation. Once the formazan was completely dissolved, the supernatant was read on a microplate reader at 590 nm and quantified against a standard containing a known number of cells. The results are reported as cells per volume of construct. All reported values are an average from three individual constructs.

Alkaline phosphatase (ALP). Alkaline phosphatase activity of cells in the HCG constructs was determined as follows. Cells were digested in 500 μl lysing buffer (1% Triton X-100, 0.5% sodium deoxycholate, 0.1% SDS, 0.1 mg/ml phenylmethylsulphonylfluoride and 3% aprotinin in PBS) for 30 min. The cell lysate (500 μl) was then added to 1 ml substrate and alkaline buffer in a dark centrifuge tube. The mixture was placed in a 37° C. water bath for 15 min, after which the reaction was stopped using 1 ml 0.5 N NaOH in PBS and read at 405 nm, using a Bio-Rad Benchmark microplate reader (Hercules, Calif., USA). p-Nitrophenol was used to construct a standard curve. Unseeded scaffolds were used as blanks. Readings were normalized by cell numbers quantified by MTT assay. All data points are an average of three replicates.

In situ ELISA. Semi-quantitative in situ ELISA assays were conducted similarly to the method outlined in Cheng et al. (2003). Briefly, samples were fixed with 2.5% glutaraldehyde, permeablized with 0.5% Triton-X and blocked with 10% goat serum and 1% bovine serum albumin in PBS. Mouse or rabbit primary antibodies, to fibronectin (FN), vitronectin (VN), osteopontin (OP) and osteocalcin (OC), and anti-mouse or anti-rabbit alkaline phosphatase (ALP)-tagged secondary antibodies were purchased from Abcam (Cambridge, Mass., USA). The samples were extensively washed in blocking buffer after incubation with each antibody to minimize non-specific binding. p-Nitrophenol phosphate ALP substrate was added and the samples were incubated at 37° C. for 30 min. The reaction was stopped with the addition of 0.5 M sodium hydroxide. The supernatant was analyzed using a Biorad Benchmark Microplate Reader (Hercules, Calif., USA) at 405 nm. Unseeded HCG scaffolds blanks were used to establish baseline, with all data points being an average of three replicates.

4′,6-Diamidino-2-phenylindole (DAPI) staining. Samples for DAPI staining were seeded with 2×10⁶ cells, using depth filtration. The cells were allowed to adhere for 6 h and then constructs were removed from the culture medium, washed briefly three times in PBS and then fixed in 2.5% glutaraldehyde in PBS for 1 h. The samples were then rinsed again in PBS, dehydrated using graded ethanol washings, immersed in three changes of xylene for 10 min each, immersed in xylene-saturated paraffin for 20 min, and finally the samples were transferred to fresh liquid paraffin overnight. The paraffin was removed and new paraffin was added; this process was repeated every 24 h for 72 h to ensure full paraffin penetration to the centre regions of the construct. The embedded samples were then cooled to room temperature, sliced to 8 μm thickness and placed on lysine-coated slides. The slides were then exposed to two xylene washes, submerged in 100% ethanol for two washes and then washed briefly in 95% ethanol, 70% ethanol and DI water, then dried. Finally, the samples were permeablized in 0.05% Triton X-100, washed with PBS and finally incubated for 30 min with DAPI and then visualized using an Olympus IX70 (Center Valley, Pa., USA) with an Optronics (Goleta, Calif., USA) camera attachment.

Statistics/data analysis. Unless otherwise noted, all experiments were performed in triplicate (n=3) and representative data with statistical significance are reported. Experimental results are expressed as mean±standard deviation (SD) of the means of samples. Statistical comparisons were performed by ANOVA for multiple comparisons, and statistical significance was accepted at p<0.05.

### Example 2-3D Porous HCG Scaffold Fabrication

The 3D HCG scaffolds were prepared by lyophilization, following an established procedure reported in our early studies (Zhao et al., 2002, 2006). An inverse correlation between pore size and freezing temperature was found to be in agreement with previous reports, indicating that the pore structure of the HCG scaffolds can be readily adjusted by regulating the freezing temperature (O'Brien et al., 2005; Zhao et al., 2002). The surface texture of the scaffolds under all freezing conditions appeared rough, with pore sizes serving as suitable templates for cell adhesion, migration and growth, varying from 99±24 μm for HCG80 scaffolds to 158±23 μm in the HCG20 scaffold (FIG. 10).

Table 2 lists the pore size of each fabricated scaffold, detailing the effect of freezing temperature on final construct pore size.

TABLE 2

| Freezing Temperature | Pore Size |
|---|---|
| −80° C. (HCG80) | 99 ± 24 μm |
| −50° C. (HCG50) | 128 ± 38 μm |
| −20° C. (HCG20) | 158 ± 23 μm |

### Example 3

### Cell Seeding and Preconditioning

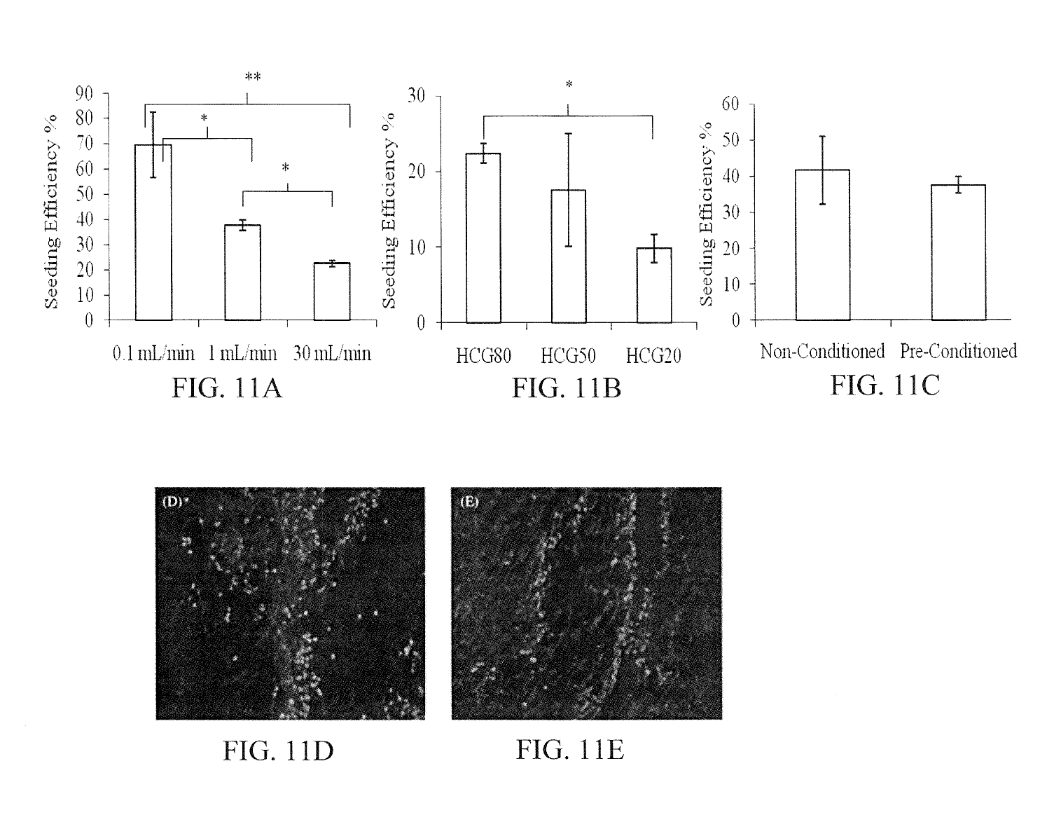

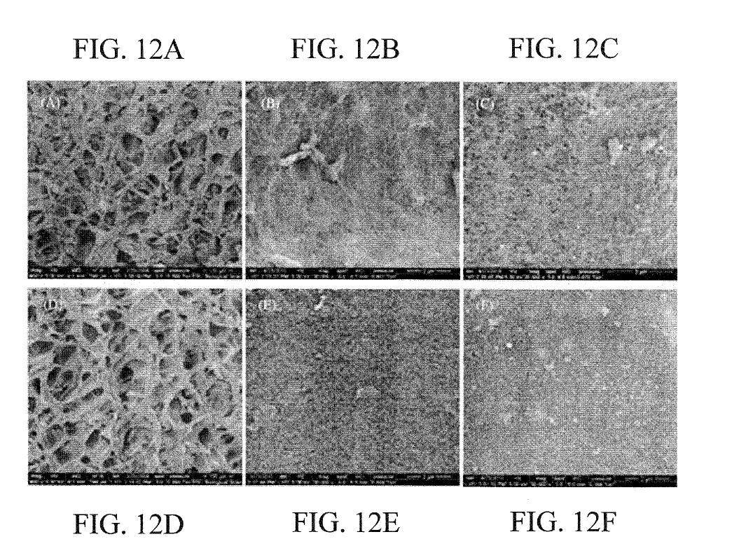

Seeding efficiency had an inverse relationship to both flow rate and pore size, as shown in FIGS. 11A and 11B. However, preconditioning of the HCG scaffolds in serum containing media had no effect on cell-seeding efficiency at 1.0 ml/min (FIG. 11C). DAPI images obtained from depth filtration 6 h after seeding at 1.0 ml/min show good cell penetration and distribution throughout the 1 mm thickness of the scaffolds (FIGS. 11D and 11E). SEM images of preconditioned samples (FIGS. 12A-12C) displayed a more fibrous surface texture after 7 days of complete medium preconditioning, giving it a less rough appearance as compared to samples stored in PBS (FIGS. 12D-12F) over the same time period. Preconditioning also induced the appearance of nanopores on the surface of the scaffold (FIG. 12C) when imaged at ×30,000. Preconditioning did not appear to effect the overall pore structure of the HCG scaffold (FIGS. 12A and 12D).

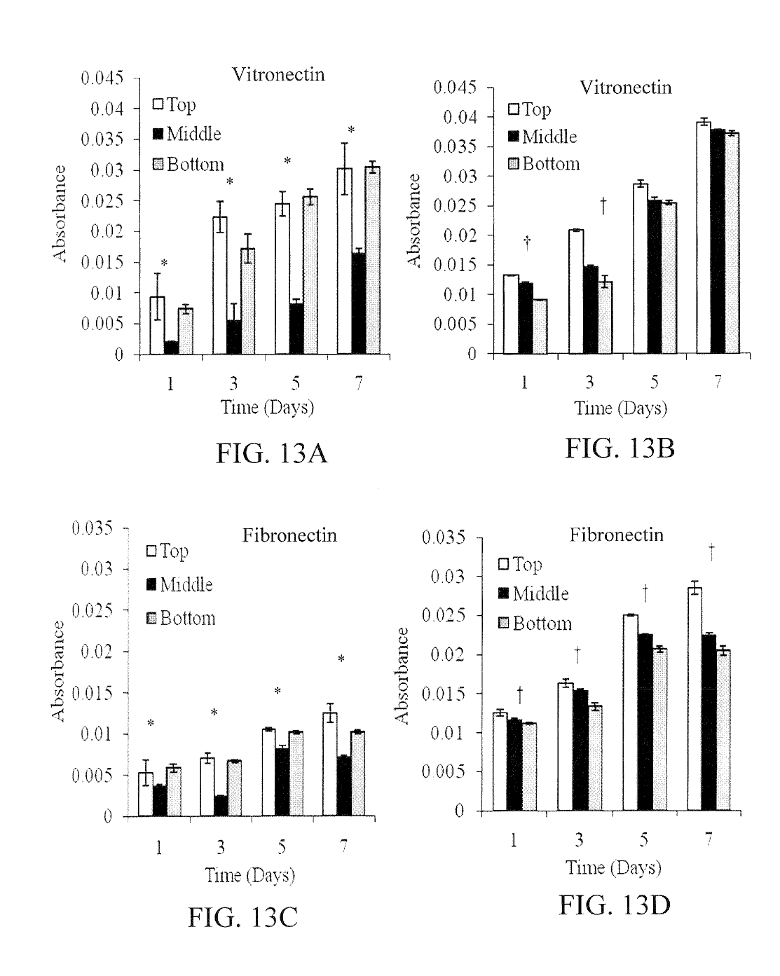

To examine the impact of perfusion conditioning on the adsorption of ECM proteins, quantitative in situ ELISA was carried out to analyze the contents of FN and VN in HCG scaffolds preconditioned under either static or perfusion conditions. There was a progressive increase of protein adsorption in both static and perfusion conditioned samples (FIG. 13), suggesting electrostatic interactions of chitosan with media proteins. However, perfusion preconditioning showed better and more homogeneous protein adsorption, with an adsorption gradient in the direction of flow at day 7 for FN. In contrast, static samples universally showed less protein adsorption and poor media protein adsorption to centre scaffolds, indicating insufficient penetration into the centre of the scaffolds (FIGS. 13A-13D). Although both FN and VN adsorption increased for the centre scaffolds over 7 days under static preconditioning, the overall adsorption levels remained about half of those scaffolds at the top and bottom, suggesting diffusion limitation under static preconditioning (FIGS. 13A and 13C). Comparison of the adsorption profiles of VN and FN further indicate that VN has a higher affinity to the HCG scaffold compared to FN and that perfusion is effective in improving VN adsorption to the middle scaffolds (FIG. 13).

### Example 4

### hMSC Proliferation and Osteogenic Differentiation

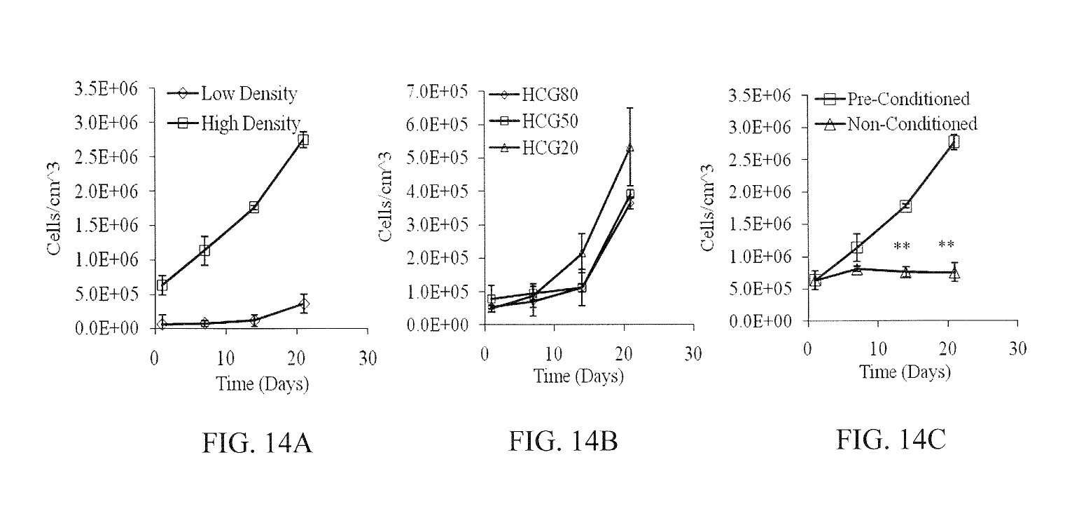

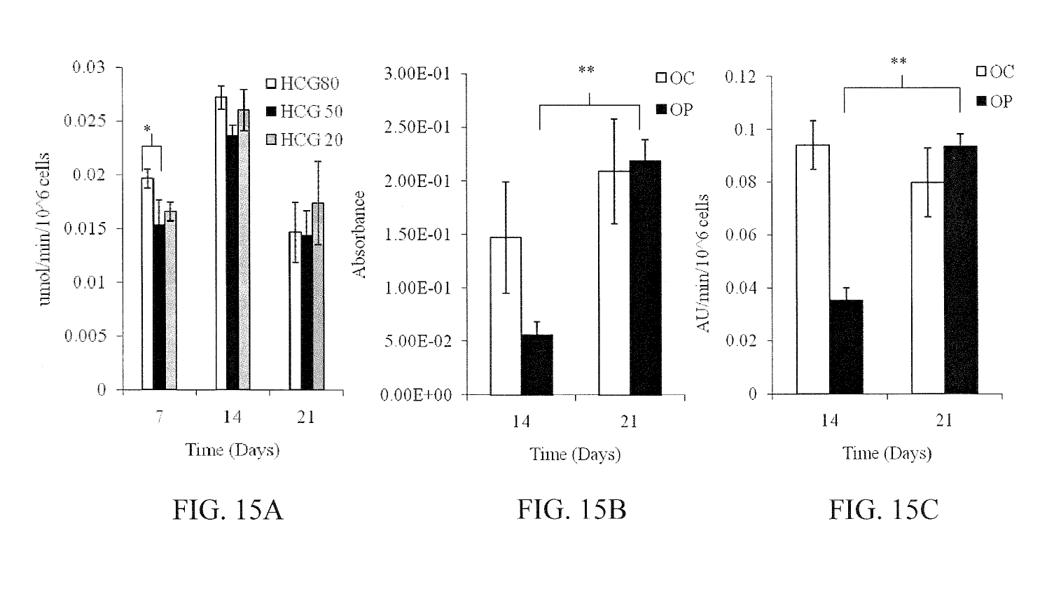

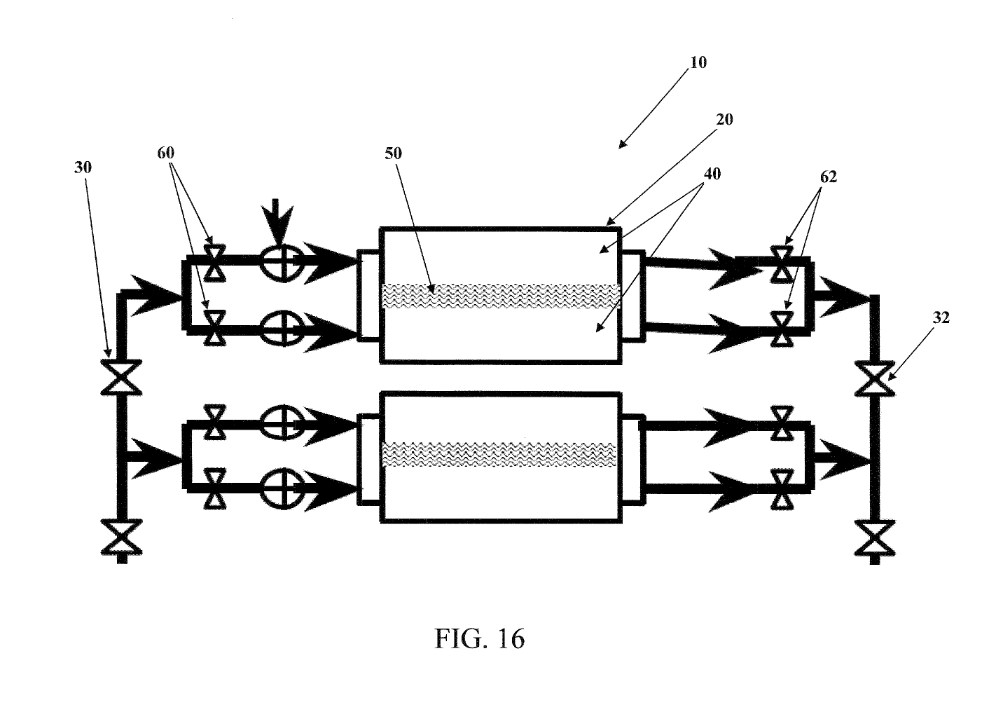

Pore size inversely influenced seeding efficiency, yet had no effect on cell growth. Although cell numbers in HCG20 appeared higher at days 14 and 21, statistical analysis indicated insignificant difference among HCG constructs with different pore sizes (FIG. 14B). Lower initial seeding density delayed hMSC proliferation by 14 days, while a higher seeding density immediately initiated cell growth, reducing the lag phase (FIG. 14A). Cells cultured at an initial lower cell density (50,000 cells/cm³) only increased in number two-fold over the first 14 days, but increased their numbers 3.2-fold between days 14 and 21 for an overall increase of 6.6-fold. In contrast, cells seeded at a higher initial cell density (500,000 cells/cm³) increased approximately 1.5-fold every 7 days for an overall increase of 4.3-fold. While preconditioning had no effect on hMSC seeding efficiency (FIG. 11C), hMSCs in preconditioned samples had more rapid growth and increased 4.3-fold over a 21 day culture period, whereas non-preconditioned samples exhibited little growth over the same culture period, only increasing 1.2-fold by day 21 (FIG. 14C).

Pore size had little effect on growth characteristics and alkaline phosphatase expression (FIG. 15A), with significantly different levels of expression being present only at day 7 between HCG80 and HCG50 (p<0.05). In situ ELISA was carried out to determine the expression of OC and OP that bind to the HCG scaffolds. OC shows no significant change in expression, but OP is significantly upregulated by over 164% (p<0.01) between days 14 and 21 (FIG. 15B).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 5,741,895
U.S. Pat. No. 6,245,899
U.S. Pat. No. 6,875,605
U.S. Pat. No. 6,943,008
U.S. Pat. No. 7,122,371
Zhao, F. (2007) Biotechnology and Bioengineering, 96(3):584-595
Yang, T. T. et al. (1996) “Optimized Codon Usage and Chromophore Mutations Provide Enhanced Sensitivity with the Green Fluorescent Protein” Nucleic Acid Research 24(22):4592-4593.
Alvarez-Barreto J F, Linehan S M, Shambaugh R L, et al. 2007; Flow perfusion improves seeding of tissue engineering scaffolds with different architectures. Ann Biomed Eng 35: 429-442.
Amaral IbF, Lamghari M, Sousa S R, et al. 2005; Rat bone marrow stromal cell osteogenic differentiation and fibronectin adsorption of chitosan membranes: the effect of degree of acetylation. J Biomed Mater Res A 75A: 387-397.
Bale M D, Wohlfahrt L A, Mosher D F, et al. 1989; Identification of vitronectin as a major plasma protein adsorbed on polymer surfaces of different co-polymer composition. Blood 74: 2698-2706.
Bernardo M E, Faffaroni N, Novara F, et al. 2007; Human bone marrow derived mesenchymal stem cells do not undergo transformation after long-term in vitro culture and do not exhibit telomere maintenance mechanisms. Cancer Res 67: 9142-9149.
Bernards M, Qin C, Ratner B, et al. 2008; Adhesion of MC3T3-E1 cells to bone sialoprotein and bone osteopontin specifically bound to collagen I. J Biomed Mater Res A 86: 779-787.
Bowman K, Leong K 2006; Chitosan nanoparticles for oral drug and gene delivery. Int'l J Nanomedicine 1(2): 117-128.
Burdick J, Vunjak-Novakovic G. 2009; Engineered microenvironments for controlled stem cell differentiation. Tissue Eng A 15: 205-219.
Caplan A. 2007; Adult mesenchymal stem cells for tissue engineering versus regenerative medicine. J Cell Physiol 213: 341-347.
Cheng M Y, Deng J G, Yang F, et al. 2003; Study on physical properties and nerve cell affinity of composite films from chitosan and gelatin solutions. Biomaterials 24: 2871-2880.
Chesnutt B, Yuan Y, Buddington K, et al. 2009; Composite chitosan/nanohydroxyapatite scaffolds induce osteocalcin production by osteoblasts in vitro and support bone formation in vivo. Tissue Eng A 15: 2571-2579.
Denhardt D, Noda M, O'Regan A, et al. 2001; Osteopontin as a means to cope with environmental insults: regulation of inflammation, tissue remodeling, and cell survival. J Clin Invest 107: 1055-1061.
Ding C M, Zhou Y, He Y H, et al. 2008; Perfusion seeding of collagen-chitosan sponges for dermal tissue engineering. Progr Biochem 43: 287-296.
Grayson W, Zhao F, Izadpanah R, et al. 2004; Human mesenchymal stem cells tissue development in 3D PET matrices. Biotechnol Prog 20: 905-912.
Helm C, Fleury E, Zisch A, et al. 2005; Synergy between interstial flow and VEGF directs capillary morphogenesis in vitro through a gradient amplification mechanism. Proc Natl Acad Sci USA 102: 15779-15784.
Horowitz E, Gordon P, Koo W, et al. 2002; Isolated allogeneic bone marrow-derived mesenchymal cells engraft and stimulate growth in children with osteogenesis imperfecta: implications for cell therapy of bone. Proc Natl Acad Sci USA 99: 8932-8937.
Horowitz E, Prockop D, Fitzpatrick L, et al. 1999; Transplantability and therapeutic effects of bone marrow-derived mesenchymal cells in children with osteogenesis imperfecta. Nat Med 5: 309-313.
Hosseinkhani H, Inatsugu Y, Hiraoka Y, et al. 2005; Perfusion culture enhances osteogenic differentiation of rat mesenchymal stem cells in collagen sponge reinforced with poly(glycolic acid) fiber. Tissue Eng 11: 1476-1488.
Hu P W, Leung P C, Sher A. 1996; Fluid conductance of cancellous bone graft as a predictor for graft-host interface healing. J. Biomech 29: 123-132.
Itoh M, Shimazu A, Hirata I, et al. 2004; Characterization of CO(3)Ap-collagen sponges using X-ray high-resolution microtomography. Biomaterials 25: 2577-2583.
Ivaska K K, Hentunen T A, Vaaraniemi J, et al. 2004; Release of intact and fragmented osteocalcin molecules from bone matrix during bone resorption in vitro. J Biol Chem 279: 18361-18369.
Jensen T, Dolatshahi-Pirouz A, Foss M, et al. 2010; Interaction of human mesenchymal stem cells with osteopontin coated hydroxyapatite surfaces. Colloids Surf B Biointerfaces 75: 186-193.
Jones A, Arms C, Hutmacher D, et al. 2009; The correlation of pore morphology, interconnectivity and physical properties of 3D ceramic scaffolds with bone ingrowth. Biomaterials 30: 1440-1451.
Kern S, Eichler H, Stoeve J, et al. 2006; Comparative analysis of mesenchymal stem cells from bone marrow, umbilical cord blood, or adipose tissue. Stem Cells 24: 1294-1301.
Kilpadi K, Chang P, Bellis S. 2001; Hydroxyapatite binds more serum proteins, purified integrins, and osteoblast precursor cells than titaniumor steel. J Biomed Mater Res 57: 258-267.
Kim H W, Kim H E, Salih V. 2006; Stimulation of osteoblast responses to biomimetic nanocomposites of gelatin-hydroxyapatite for tissue engineering scaffolds. Biomaterials 26: 5221-5230.
Li Y, Ma T, Kniss D A, et al. 2001; Effects of filtration seeding on cell density, spatial distribution, and proliferation in nonwoven fibrous matrices. Biotechnol Prog 17: 935-944.
Murphy C M, Haugh M G, O'Brien F J. 2010; The effect of mean pore size on cell attachment, proliferation and migration in collagen-glycosaminoglycan scaffolds for bone tissue engineering. Biomaterials 31: 461-466.
Ng C, Helm L, Swartz M. 2004; Interstitial flow differentially stimulates blood and lymphatic endothelial cell morphogenesis. Microvasc Res 68: 258-264.
O'Brien F, Harley B, Yannas I, et al. 2005; The effect of pore size on cell adhesion in collagen-GAG scaffolds. Biomaterials 26: 433-441.
Peter M, Ganesh N, Selvamurugan N, et al. 2010; Preparation and characterization of chitosan-gelatin/nanohydroxyapatite composite scaffolds for tissue engineering applications. Carbohydr Polym 80: 687-694.
Roy T D, Simon J L, Ricci J L, et al. 2003; Performance of degradable composite bone repair products made via three-dimensional fabrication techniques. J Biomed Mater Res A 66A: 283-291.
Salgado A J, Oliveira J T, Pedro A J, et al. 2006; Adult stem cells in bone and cartilage tissue engineering. Curr Stem Cell Res Ther 1: 345-364.
Seib F P, Prewitz M, Werner C, et al. 2009; Matrix elasticity regulates the secretory profile of human bone marrow multipotent mesenchymal stromal cells (MSCs). Biochem Biophys Res Commun 389: 663-667.
Semino C, Kamm R, Lauffenburger D. 2006; Autocrine EGF receptor activation mediates endothelial cell migration and vascular morphogenesis induced by VEGF under interstitial flow. Exp Cell Res 312: 289-298.
Sikavitsas V I, Bancroft G N, Holtorf H L, et al. 2003; Mineralized matrix deposition by marrow stromal osteoblasts in 3D perfusion culture increases with increasing fluid shear forces. Proc Natl Acad Sci USA 100: 14683-14688.
Sionkowska A, Wisniewski M, Skopinska J, et al. 2004; Molecular interactions in collagen and chitosan blends. Biomaterials 25: 795-801.
Sundelacruz S, Kaplan D. 2009; Stem cell and scaffold-based tissue engineering approaches to osteochondral regenerative medicine. Semin Cell Dev Biol 20: 646-655.
Takahashi Y, Tabata Y. 2004; Effect of fiber diameter and porosity on non-woven PET fabrics on the osteogenic differentiation of mesenchymal stem cells. J Biomater Sci Polymer Ed 15: 41-57.
Taravel M, Domard A. 1996; Collagen and its interactions with chitosan III. Some biological and mechanical properties. Biomaterials 17: 451-455.
Tseng S, Lee M, Reddi A. 2008; Nonunions and the potential of stem cells in fracture healing. J Bone Joint Surg 90A: 92-98.
Uygun B E, Bou-Akl T, Albanna M, et al. 2010; Membrane thickness is an important variable in membrane scaffolds: influence of chitosan membrane structure on the behavior of cells. Acta Biomater 6: 2126-2131.
Weinreb M, Shinar D, Rodan G. 1990; Different pattern of alkaline phosphatase, osteopontin, and osteocalcin expression in developing rat bone visualized by in situ hybridization. J Bone Miner Res 5: 831-842.
Whyte M, Kurtzberg J, McAlister W, et al. 2003; Marrow cell transplantation for infantile hypophosphatasia. J Bone Miner Res 18: 92-98.
Yamane S, Iwasaki N, Kasahara Y, et al. 2007; Effect of pore size on in vitro cartilage formation using chitosan-based hyaluronic acid hybrid polymer fibers. J Biomed Mater Res 81A: 586-593.
Zhao F, Grayson W L, Ma T, et al. 2006; Effects of hydroxyapatite in 3D chitosan-gelatin polymer network on human mesenchymal stem cell construct development. Biomaterials 27: 1859-1867.
Zhao F, Ma T. 2005; Perfusion bioreactor system for human mesenchymal stem cell tissue engineering: dynamic cell seeding and construct development. Biotechnol Bioeng 91: 482-493.
Zhao F, Grayson W L, Ma T, et al. 2009; Perfusion affects the tissue development patterns of human mesenchymal stem cells in 3D scaffolds. J Cell Physiol 219: 421-429.
Zhao F, Yin Y, Lu W W, et al. 2002; Preparation and histological evaluation of biomimetic three-dimensional hydroxyapatite-chitosan-gelatin network composite scaffolds. Biomaterials 23: 3227-3234.

I claim:

1. A porous hydrogel or three-dimensional (3D) scaffold that is capable of supporting growth and/or differentiation of a cell.

2. The porous hydrogel or 3D scaffold according to claim 1, wherein said scaffold comprises a hydroxyapatite-chitosan-gelatin (HCG) compound.

3. The porous hydrogel or 3D scaffold according to claim 1, wherein said scaffold is impregnated or coated with a nucleic acid encoding one or more proteins.

4. The porous hydrogel or 3D scaffold according to claim 3, wherein said nucleic acid is provided in a nanoparticle that encapsulates said nucleic acid.

5. The porous hydrogel or 3D scaffold according to claim 4, wherein said nanoparticle is a chitosan-based nanoparticle.

6. The porous hydrogel or 3D scaffold according to claim 3, wherein said nucleic acid encodes a protein associated with growth and/or differentiation of a cell to a desired tissue type.

7. The porous hydrogel or 3D scaffold according to claim 3, wherein said nucleic acid encodes a bone morphogenetic protein (BMP).

8. The porous hydrogel or 3D scaffold according to claim 7, wherein said BMP is BMP-1, BMP-2, BMP-3, BMP-4, BMP-5, BMP-6, BMP-7, BMP-8a, BMP-8b, BMP-9, BMP-10, or BMP-15.

9. The porous hydrogel or 3D scaffold according to claim 1, wherein said nucleic acid encodes one or more of a fibroblast growth factor (FGF), a vascular endothelial growth factor (VEGF), a transforming growth factor-beta (TGF-β) or a cell density signal molecule.

10. The porous hydrogel or 3D scaffold according to claim 1, wherein said nucleic acid is provided in a nucleic acid expression construct.

11. The porous hydrogel or 3D scaffold according to claim 1, wherein said hydrogel or 3D scaffold comprises a stem cell.

12. The porous hydrogel or 3D scaffold according to claim 11, wherein said stem cell is a mesenchymal stem cell (MSC).

13. The porous hydrogel or 3D scaffold according to claim 1, wherein the diameter of a pore is about 75 μm to about 250 μm.

14. The porous hydrogel or 3D scaffold according to claim 1, wherein said hydrogel or scaffold is preconditioned with a fluid media that supports cell growth and/or differentiation.

15. The porous hydrogel or 3D scaffold according to claim 14, wherein said fluid media comprises serum.

16. The porous hydrogel or 3D scaffold according to claim 14, wherein the preconditioning comprises continuously circulating said fluid media through said hydrogel or scaffold at a selected flow rate for a selected period of time.

17. The porous hydrogel or 3D scaffold according to claim 1, wherein said hydrogel or 3D scaffold comprises a chondrocyte and/or an osteoblast.

18. A perfusion bioreactor comprising one or more perfusion chambers that can be individually controlled, and wherein transverse or parallel flow of a fluid media can be provided to each chamber, wherein said fluid provides conditions for cell life and/or supports and directs growth and/or differentiation of a cell, and wherein said chamber has two or more compartments connected by a porous hydrogel or 3D scaffold that is capable of supporting growth and/or differentiation of a cell as defined in claim 1, wherein the parallel flow rate and composition of said fluid media in each compartment of each of said chambers can be individually controlled.

19. A method for growing and/or differentiating cells or tissue, said method comprising seeding said cells in a porous hydrogel or 3D scaffold that is capable of supporting growth and/or differentiation of a cell and providing said cell-seeded porous hydrogel or 3D scaffold in a perfusion bioreactor of claim 18 under conditions that support growth and/or differentiation of said cells or tissue.

20. The method according to claim 19, wherein said scaffold comprises a hydroxyapatite-chitosan-gelatin (HCG) compound.

21. The method according to claim 19, wherein said scaffold is impregnated or coated with a nucleic acid encoding one or more proteins, wherein said cells are transfected by said nucleic acid.

22. The method according to claim 21, wherein said nucleic acid is provided in a nanoparticle that encapsulates said nucleic acid.

23. The method according to claim 22, wherein said nanoparticle is a chitosan-based nanoparticle.

24. The method according to claim 21, wherein said nucleic acid encodes a protein associated with growth and/or differentiation of a cell to a desired tissue type.

25. The method according to claim 21, wherein said nucleic acid encodes a bone morphogenetic protein (BMP).

26. The method according to claim 25, wherein said BMP is BMP-1, BMP-2, BMP-3, BMP-4, BMP-5, BMP-6, BMP-7, BMP-8a, BMP-8b, BMP-9, BMP-10, or BMP-15.

27. The method according to claim 19, wherein said cells are stem cells.

28. The method according to claim 27, wherein said stem cells are mesenchymal stem cells (MSCs).

29. The method according to claim 19, wherein said cells are differentiated into chondrocytes and/or osteoblasts.

30. The method according to claim 21, wherein said nucleic acid is provided in a nucleic acid expression construct.

31. The method according to claim 19, wherein said porous hydrogel or 3D scaffold is in the shape and/or size of a tissue that is to be implanted in a person or animal.
